SEQUENCE LISTING (19 sequences)

SEQ ID NO: 1 - DNA, 18 bp, Artificial Sequence
  PCR primer
tcgccattcaggctgcgc

SEQ ID NO: 2 - DNA, 18 bp, Artificial Sequence
  PCR primer
tgttcgggttgctgatgc

SEQ ID NO: 3 - DNA, 18 bp, Artificial Sequence
  PCR primer
cctcttcgctattacgcc

SEQ ID NO: 4 - DNA, 18 bp, Artificial Sequence
  PCR primer
tgaccatccggaactgtg

SEQ ID NO: 5 - DNA, 30 bp, Artificial Sequence
  PCR primer
ccaaactggaacaacactcaaccctatctc

SEQ ID NO: 6 - DNA, 25 bp, Artificial Sequence
  PCR primer
gtcagtgagcgaggaagcggaagag

SEQ ID NO: 7 - DNA, 27 bp, Artificial Sequence
  PCR primer
cgattaagttgggtaacgccagggttt

SEQ ID NO: 8 - DNA, 25 bp, Artificial Sequence
  PCR primer
agctcactcattaggcaccccaggc

SEQ ID NO: 9 - DNA, 18 bp, Artificial Sequence
  PCR primer
tccattgcgcatcgttgc

SEQ ID NO: 10 - DNA, 18 bp, Artificial Sequence
  PCR primer
agtactagttcgaacgcg

SEQ ID NO: 11 - DNA, 18 bp, Artificial Sequence
  PCR primer
acagcgttgttccactgg

SEQ ID NO: 12 - DNA, 18 bp, Artificial Sequence
  PCR primer
aagctgcatcagcttcag

SEQ ID NO: 13 - DNA, 17 bp, Artificial Sequence
  Donor plasmid terminal repeat sequence
tgattatctttctaggg

SEQ ID NO: 14 - DNA, 26 bp, Artificial Sequence
  Chinese Hamster Ovary (CHO) chromosomal sequence
gctcgggccggccgcgtcgccgcttc

SEQ ID NO: 15 - DNA, 26 bp, Artificial Sequence
  Chinese Hamster Ovary (CHO) chromosomal sequence
caatcaataagataaacatacacaga

SEQ ID NO: 16 - DNA, 26 bp, Artificial Sequence
  Chinese Hamster Ovary (CHO) chromosomal sequence
caccacatttaacttgctctttgata

SEQ ID NO: 17 - DNA, 26 bp, Artificial Sequence
  Chinese Hamster Ovary (CHO) chromosomal sequence
tagagtgctgagatttgggacattgc

SEQ ID NO: 18 - DNA, 26 bp, Artificial Sequence
  Chinese Hamster Ovary (CHO) chromosomal sequence
ggcgttggtggcacacaactttaagt

SEQ ID NO: 19 - DNA, 26 bp, Artificial Sequence
  Chinese Hamster Ovary (CHO) chromosomal sequence
taagacaatgtatgactttgtcccat

(19) United States
(12) Patent Application Publication
(10) Pub. No.: US 2011/0047635 A1
(43) Pub. Date: Feb. 24, 2011

(54) METHODS AND COMPOSITIONS FOR TRANSPOSON-MEDIATED TRANSGENESIS

(75) Inventors: Stefan Moisyadi, Honolulu, HI (US); Joseph M. Kaminski, Evans, GA (US); Kazuto Morozumi, Fukushima (JP); Pawel Pelczar, Egg (CH)

Correspondence Address: DAVIS WRIGHT TREMAINE LLP - San Francisco, 505 MONTGOMERY STREET, SUITE 800, SAN FRANCISCO, CA, 94111, US

(73) Assignee: University of Hawail, Oonolulu, HI (US)

(21) Appl. No.: 12/438,519

(22) PCT Filed: Aug. 28, 2007
(86) PCT No.: PCT/US2007/018922

§ 371 Date: Nov. 8, 2010

Related U.S. Application Data
(60) Provisional application No. 60/840,833, filed on Aug. 28, 2006.
(60) Provisional application No. 60/840,780, filed on Aug. 28, 2006.
(60) Provisional application No. 60/859,652, filed on Nov. 17, 2006.

(51) Int. Cl. A01K 67/00; C12N 15/87

(52) U.S. Cl. 800/25

(57) ABSTRACT

Methods and compositions for transposon-mediated transgenesis are provided herein. In some embodiments, methods are provided for generating a transgenic embryo containing a piggyBac-like transposon. In some embodiments, such methods can include: contacting a nucleic acid containing a transgene flanked by two terminal repeats with one of the group consisting of: a piggyBac-like transposase polypeptide and a nucleotide sequence encoding a piggyBac-like transposase to form a mixture; contacting the mixture with a sperm to form a composition; and introducing the composition into an unfertilized oocyte to form a transgenic embryo, wherein the piggyBac-like transposase catalyzes the integration of the transgene into the genome of the embryo. In some embodiments, the piggyBac-like transposase can be encoded by a nucleotide sequence on the same nucleic acid containing the transgene. In some embodiments, the nucleic acid encoding the piggyBac-like transposase can be an mRNA.

### CROSS-REFERENCE TO RELATED APPLICATIONS

The present application claims the benefit of the filing date of U.S. Provisional Patent Application Ser. Nos. 60/840,780, filed on Aug. 28, 2006, 60/840,833, filed on Aug. 28, 2006, and 60/859,652, filed on Nov. 17, 2006, each of which is hereby incorporated by reference in its entirety.

### STATEMENT REGARDING FEDERALLY SPONSORED RESEARCH OR DEVELOPMENT

This invention was made in part with Government support under IDeA Network of Biomedical Research Excellence/National Institutes of Health Grant RR016467-06. The Government has certain rights in the invention.

### FIELD

The present invention relates to methods for the generation of transgenic cells and animals. Particular embodiments relate to the use of transposases, transposons, their nature and the modes of use by which they effectively generate transgenic cells and animals. Further embodiments relate to kits and transgenic animals useful for practicing such methods.

### BACKGROUND

The generation of transgenic animals and cells has great value in both basic and applied genetic research and in commercial applications. Transgenesis relies on the integration of exogenous nucleic acid into a host cell. Integration can be achieved passively, where insertion of a transgene is mediated by host cell DNA repair mechanisms. However, passive transgenesis occurs at a very low frequency. Transgenesis can also be performed in an active manner, using viruses and viral-based vectors that encode DNA-integrating components. Such methods produce higher frequencies of transgene insertion, but introduce risks associated with the use of attenuated or inactivated viruses and viral vectors. The most significant obstacle to the use of viral and other transgenesis systems in gene therapy and genetic research, however, is the random nature of gene insertion. This randomness introduces the risk of insertional mutagenesis. Such risks are typified by the activation of oncogenes and inactivation of tumor-repressor genes observed in mice and the development of leukemias in patients in limited human gene therapy trials. Thus, wider application of transgenic technology will require the development of active transgenesis methods that provide efficient gene integration at nonrandom sites in the genome.

### SUMMARY

Methods and compositions for transposon-mediated transgenesis are provided herein. In some embodiments, methods are provided for generating a transgenic embryo containing a piggyBac-like transposon. In some embodiments, such methods can include: contacting a nucleic acid containing a transgene flanked by two terminal repeats with one of the group consisting of: a piggyBac-like transposase polypeptide and a nucleotide sequence encoding a piggyBac-like transposase to form a mixture; contacting the mixture with a sperm to form a composition; and introducing the composition into an unfertilized oocyte to form a transgenic embryo, wherein the piggyBac-like transposase catalyzes the integration of the transgene into the genome of the embryo. In some embodiments, the piggyBac-like transposase can be encoded by a nucleotide sequence on the same nucleic acid containing the transgene. In some embodiments, the nucleic acid encoding the piggyBac-like transposase can be an mRNA.

In some embodiments, the transgene can be under the control of a promoter. In some embodiments, the transgene can be under the control of the CAG promoter. In some embodiments, the piggyBac-like transposase can be a chimeric transposase can include a host-specific DNA binding domain. In some embodiments, the host-specific DNA binding domain of the chimeric transposase can include Gal4 ZFP. In some embodiments, the host-specific DNA binding domain of the chimeric transposase can be optimized for host specificity. In some embodiments, transgene can include a selectable marker or reporter gene, inclduing, for example, EGFP, luciferase, β-galactosidase, kanamycin resistance gene (neomycin phosphotransferase), hygromycin resistance gene (hygromycin phosphotransferase), R6K gamma ori, and the like. In some embodiments, the host-specific DNA binding domain of the chimeric transposase can be fused to the N-terminus of the transposase. Likewise, in some embodiments, the host-specific DNA binding domain of the chimeric transposase can be fused to the C-terminus of the transposase.

In some embodiments, there are provided methods for generating a transgenic animal. The methods can include implanting, into a viable mother, an embryo generated according to any of the methods of the invention. In some embodiments, the mother can be a vertebrate.

In some embodiments, there are provided methods for generating a transgenic animal, containing in the genome of one or more of its cells a piggyBac-like transposon. The methods can include: contacting a nucleic acid containing a transgene flanked by two terminal repeats, with a sperm to form a mixture; introducing the mixture into an unfertilized oocyte from a transgenic female containing in its genome a piggyBac-like transposon encoding a piggyBac-like transposase under the control of an oocyte developmental promoter, thus forming a transgenic embryo, whereby the transposase can be expressed in the oocyte and catalyzes the integration of the transgene into the genome of the embryo; and implanting the transgenic embryo into a viable mother. Some embodiments of the invention include methods of generating a transgenic animal including the steps of: contacting with a sperm a nucleic acid including a transposable exogenous nucleotide sequence and a nucleotide sequence encoding a transposase on the same nucleic acid; and introducing the nucleic acid contacted with the sperm into an oocyte to form a transgenic embryo, whereby the transposase is expressed in the embryo and catalyzes integration of the transposable exogenous nucleotide sequence into the genome of the embryo.

Other embodiments of the invention include methods of generating a transgenic animal including the steps of: incubating a mixture of a transposable exogenous nucleic acid and a nucleic acid encoding a transposase; contacting the mixture with a sperm; introducing the mixture with the sperm into an oocyte to form a transgenic embryo, whereby the transposase is expressed in the embryo and catalyzes integration of the transposable exogenous nucleotide sequence into the genome of the embryo. In some embodiments, the nucleic acid encoding the transposase is an mRNA.

Further embodiments are directed to methods of generating a transgenic animal including the steps of: incubating a mixture of a transposable exogenous nucleic acid with a transposase polypeptide; contacting the mixture with a sperm; and introducing the mixture with the sperm into an oocyte to form a transgenic embryo, whereby the transposase catalyzes integration of the transposable exogenous nucleotide sequence into the genome of the embryo.

Exogenous nucleic acids, sperm, pollen, male gametes, sperm heads, oocytes, ova, female gametes, and the like, obtained from any suitable animal including vertebrates, invertebrates, plants, mammals, fish, amphibians, reptiles, birds, rodents, cats, dogs, cows, pigs, sheep, goats, horses, primates, and the like, are useful in the invention. Embodiments of the present invention include transposable exogenous nucleic acids that are flanked by nucleic acid sequences to form an inverted repeat sequence recognized by a transposase. The exogenous nucleic acid may contain more than one transgene and/or more than one transposable exogenous sequence. Prokaryotic and eukaryotic transposases are useful in the present invention. Embodiments of the invention also encompass chimeric transposases each including a host-specific DNA binding domain.

Further aspects of the invention relate to methods of generating a transgenic animal by introducing a nucleic acid, including a transposable exogenous nucleotide sequence and a nucleotide sequence encoding a transposase on the same nucleic acid, into an in vitro fertilized (IVF) oocyte to form a transgenic embryo, whereby the transposase is expressed in the embryo and catalyzes integration of the transposable exogenous nucleotide sequence into the genome of the embryo.

Other aspects of the invention relate to methods of generating a transgenic animal including the steps of: incubating a mixture of a transposable exogenous nucleic acid and a nucleic acid encoding a transposase; and introducing the mixture into an in vitro fertilized (IVF) oocyte to form a transgenic embryo, whereby the transposase is expressed in the embryo and catalyzes integration of the transposable exogenous nucleotide sequence into the genome of the embryo. In some embodiments, the nucleic acid encoding the transposase is an mRNA.

Further embodiments are directed to methods of generating a transgenic animal including the steps of: incubating a mixture of a transposable exogenous nucleic acid with a transposase polypeptide; and introducing the mixture into an in vitro fertilized (IVF) oocyte to form a transgenic embryo, whereby the transposase catalyzes integration of the transposable exogenous nucleotide sequence into the genome of the embryo.

Further embodiments relate to methods of generating a transgenic animal including the steps of: contacting with a round spermatid a nucleic acid including a transposable exogenous nucleotide sequence and a nucleotide sequence encoding a transposase on the same nucleic acid; and introducing the nucleic acid contacted with the spermatid into an artificially activated oocyte to form a transgenic embryo, whereby the transposase is expressed in the embryo and catalyzes integration of the transposable exogenous nucleotide sequence into the genome of the embryo. Other embodiments include methods wherein the transposable exogenous nucleotide sequence and the nucleotide sequence encoding a transposase are on different nucleic acids. In still further embodiments, the transposable exogenous nucleic acid is introduced with a transposase polypeptide.

Embodiments of the invention further encompass methods of generating a transgenic animal including the steps of contacting with a sperm a transposable exogenous nucleic acid, and introducing the nucleic acid and the sperm into an oocyte isolated from a transgenic animal including in its genome a transposon encoding a transposase under the control of an oocyte developmental promoter, thus forming a transgenic embryo wherein the transposase catalyzes integration of the transposable exogenous nucleotide sequence into the genome of the embryo.

Yet further embodiments relate to methods of generating a recombinant animal cell in culture including the steps of introducing into an animal cell in culture a transposable exogenous nucleic acid, and, within the same or on a separate nucleic acid, a nucleotide sequence encoding a transposase, and culturing the cell under conditions in which the transposase is expressed in the cell and catalyzes integration of the transposable exogenous nucleotide sequence into the genome of the cell. In certain embodiments, the nucleic acid encoding the transposase is a separate nucleic acid. In some embodiments, the separate nucleic acid encoding the transposase is an mRNA.

Certain embodiments relate to methods of generating a recombinant animal cell in culture including the steps of introducing into a cell in culture a transposable exogenous nucleic acid and a transposase polypeptide, and culturing the cell under conditions in which the transposase catalyzes integration of the transposable exogenous nucleotide sequence into the genome of the cell.

Embodiments of the invention encompass methods to generate transgenic animals wherein the transgenic embryo is implanted into a surrogate mother of the same species under conditions that favor the development of the transgenic embryo into a transgenic offspring. Reagents useful in the embodiments include, for example, unfertilized metaphase II stage oocytes, in vitro fertilized (IVF) oocytes, artificially activated oocytes, ova, spermatozoa, spermatids, sperm heads, membrane-disrupted sperm, pollen, demembranated sperm, and the like. Methods for introducing components of the embodiments, such as the transposable exogenous nucleic acid, transposase, and sperm head into an oocyte include, for example, microinjection, intracytoplasmic sperm injection (ICSI), pronuclear microinjection, particle bombardment, electroporation, lipid vesicle transfection, and the like. Methods for introducing components of the embodiments into an animal cell in culture include, for example, microinjection, particle bombardment, electroporation, lipid vesicle transfection, and the like.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1 depicts the plasmid designated pMMK-1 containing both the transposase gene and the transposon construct, including between its 5′ and 3′ terminal repeats (TRs) the gene for EGFP driven by the CAG promoter and the pSV40-hygromycin and ColE1 kanamycin resistance genes. The piggyBac transposase gene is driven by the CAG promoter.

FIG. 2 depicts a new plasmid designated pMMK-2, similar to pMMK-1, but containing the piggyBac transposase gene driven by the CMV promoter and containing two kanamycin resistance genes.

FIG. 3 illustrates the enhanced efficiency of transgenesis in human HEK293 cells transfected with the pMMK-1 plasmid (left) relative to cells transfected with a control plasmid lacking the piggyBac transposase gene (right).

FIG. 4 shows representations of two-plasmid transposon systems used to directly compare the genomic integration efficiencies of piggyBac and three other transposases. Each transposase was encoded on a helper plasmid (A), each of which was cotransfected into cultured mammalian cells along with a donor plasmid (B). The number of cell clones resistant to the antibiotic hygromycin was then measured to reveal the efficiency of genomic insertion of the pSV40-hygromycin resistance gene on the donor plasmid by each of the transposases.

FIG. 5 shows the high transposition activity of piggyBac transposase relative to three other transposases, Sleeping Beauty (SB11), Mos1, and Tol2, in four different mammalian cell lines, (A) HeLa, (B) H1299, (C) HEK293, and (D) CHO cells, each transfected with the plasmids from FIG. 4. (E) An example of hygromycin-resistant HEK293 cells transfected with piggyBac, Tol2, and SB11 transposon systems (from left to right), and their controls, stained for visibility. (F) PCR-based detection of transposon sequences excised from the donor plasmid in vivo by the transposase indicated, showing excision by SB11 (left) and piggyBac (middle), but not by Mos1 (right).

FIG. 6 depicts the transposition activity of SB11 (A), Tol2 (B), and piggyBac (C and D) at various ratios of helper to donor plasmid. The activities of SB11 and piggyBac both peak at certain ratios and then decrease as the amount of helper plasmid increases. Tol2 does not exhibit such overproduction inhibition, and its activity continues to rise as helper plasmid is increased.

FIG. 7 depicts the transposition activity of chimeric transposases containing N-terminal GAL4 DNA binding domains (A). GAL4-piggyBac retains the activity of its non-chimeric, wild type counterpart, while GAL4-SB11 and GAL4-Tol2 have negligible activity (B).

FIG. 8 depicts a diagram of transgenesis using chimeric transposon technology (CU). A single plasmid encoding a transgene and a chimeric, site-selective transposase, both under the control of an enhancer and promoter, is transfected into a cell. The transposase becomes expressed, binds the terminal repeats flanking the transgene, excises and directs insertion of the transgene into a specific site in the cell's genomic DNA.

FIG. 9 depicts transgenic mice pups examined for EGFP expression in their skin by epifluorescence following transgenesis using pMMK-2.

FIG. 10 depicts a plasmid encoding piggyBac transposase used in the construction of plasmid pMMK-1 (FIG. 1).

FIG. 11 depicts a plasmid containing a piggyBac transposon used in the construction of plasmids pMMK-1 (FIG. 1) and pMMK-2 (FIG. 2).

FIG. 12 depicts a plasmid encoding piggyBac transposase used in the construction of plasmid pMMK-2 (FIG. 2).

FIG. 13 depicts a “transposome” complex formed by a purified transposase similar to piggyBac, the Tn5 transposase, and a Tn5 transposon-donating plasmid.

FIG. 14 depicts a control (c), nontransgenic mouse and a transgenic mouse (t) expressing EGFP after transgenesis using a Tn5 transposome.

FIG. 15 depicts Southern blot analysis revealing copy numbers of a transgene in mice generated from Tn5-transposome-injected embryos, as well as an image of an EGFP-transgenic and nontransgenic mouse.

FIG. 16 depicts PCR and Southern blot testing for transposon integration in transgenic mice generated using a Tn5-transposome.

FIG. 17 depicts the schematics of an interplasmid transposition assay used to test the activity of chimeric Mos1 and piggyBac transposases.

FIG. 18 depicts a map of a target plasmid for transgene insertion by a chimeric Mos1 transposase.

FIG. 19 depicts a map of a target plasmid for transgene insertion by a chimeric piggyBac transposase.

### DETAILED DESCRIPTION

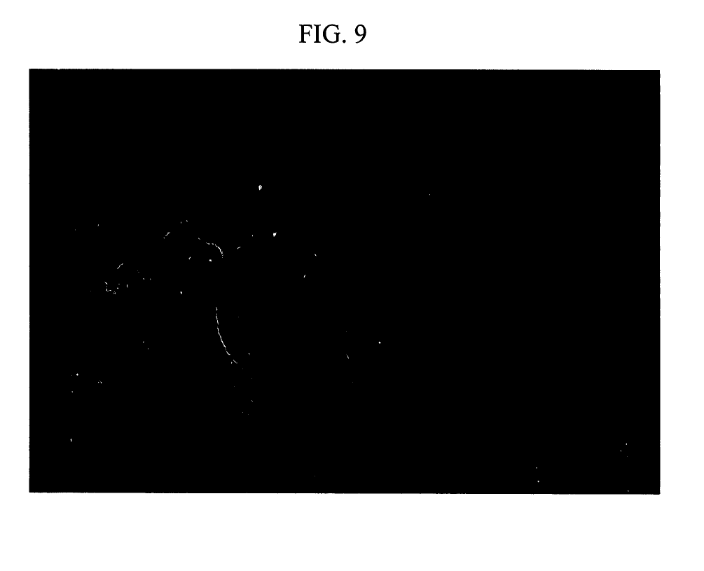

Embodiments of the invention relate to the discovery that transgenic animals can be produced by microinjecting a nucleic acid containing both a transposable exogenous nucleotide sequence and a sequence encoding a piggyBac transposase, along with a sperm head, into the cytoplasm of an unfertilized metaphase II oocyte to form a transgenic embryo, whereby the transposase catalyzes integration of the transposable exogenous nucleotide sequence into the genome of the transgenic embryo; implanting the transgenic embryo into a surrogate mother and allowing the transgenic embryo to develop into a transgenic offspring (See FIG. 9 and Table 1). Transgenic animals and plants have many uses including genetic research, gene therapy, crop and animal improvement, and producing therapeutic and non-therapeutic molecules. Embodiments of the invention provide methods for generating transgenic embryos, animals, and plants. Embodiments of the invention encompass transposase-mediated transgenesis methods including transposase-mediated intracytoplasmic sperm injection (TN:ICSD, transposase-mediated intracytoplasmic round spermatid injection (TN:ROSI), and transposase-mediated in vitro fertilization (TN:IVF). The use of chimeric transposase enzymes to catalyze site-selective integration of the exogenous nucleotide sequence into the genome of the transgenic embryo is also comprehended.

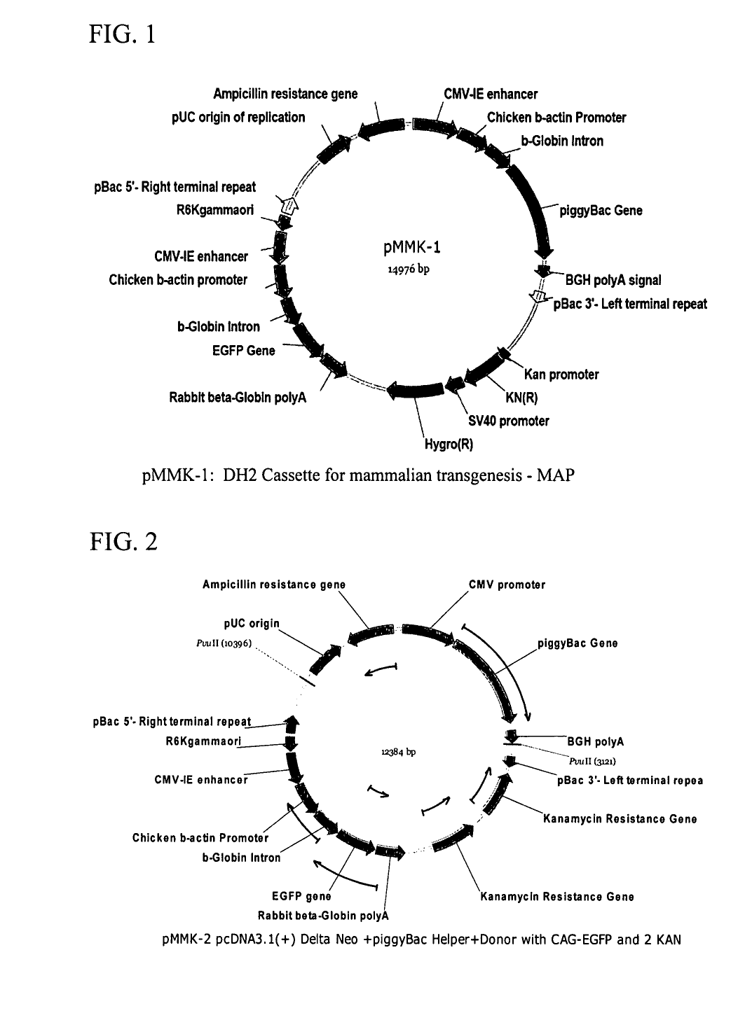

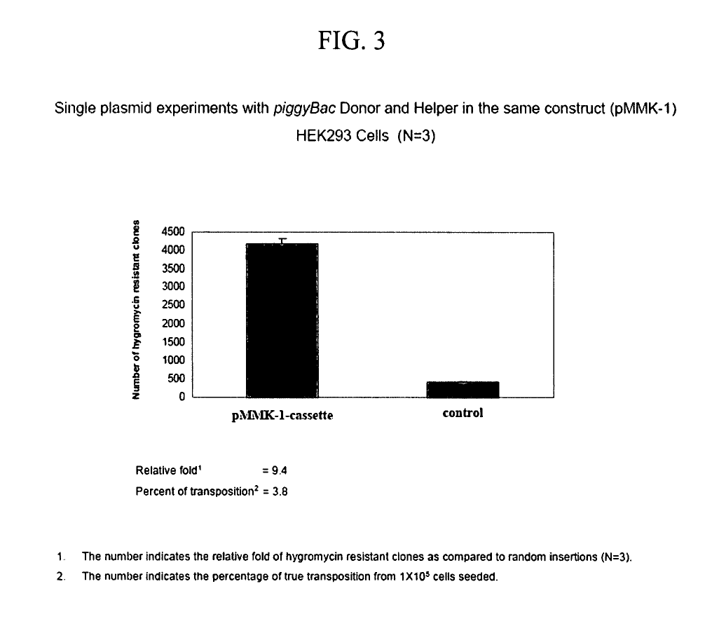

DNA level transposase enzymes discovered to date that have been applied to transgenic or gene therapy attempts work as a two plasmid system: (1) a helper plasmid that expresses the transposase, and (2) a donor plasmid that contains the transposon. Such systems, including the transposase known as piggyBac, are described as having a helper plasmid expressing the transposase in the “trans” position to the donor plasmid (U.S. Pat. No. 6,962,810). PiggyBac is the most effective transposase for transforming human cell lines when compared head to head with other transposases commonly used (SleepingBeauty [SB11], Tol2 and Mos1) (Wu, S. C.-Y., et al., P.N.A.S. [2006] 103[41]:15008-15013). Construction of a single plasmid can be achieved by joining the helper and donor plasmids of the piggyBac system and eliminating the redundant sequences in the original helper and donor constructs. This new plasmid, designated pMMK-1, (FIG. 1) contains both the transposase gene and the transposon construct. Included between the 5′- and 3′-terminal repeats (TRs) of the tranposon sequence is the gene for EGFP driven by the CAG promoter as well as the pSV40-hygromycin and ColE1 kanamycin resistance genes. The piggyBac transposase gene is also driven by the CAG promoter in pMMK-1. The plasmid pMMK-2 (FIG. 2) is similar to pMMK-1, but the transposase gene is driven by the CMV promoter instead of the CAG promoter. Transfection of HEK293 cells with pMMK-1 is 10-fold more frequent than cells transfected with control plasmid that lacks the piggyBac gene (FIG. 3 and Example 2). Such results are encouraging for potential use of this plasmid in gene therapy experiments.

Studies describing transposition by piggyBac transposase systems to date have supplied piggyBac-encoding plasmid DNA as the source of the transposase. However, for transgenesis in plants and animals, including human gene therapies, the transposase-encoding DNA sequence, when delivered into cells, can itself become integrated into the host genome via transposase-independent non-homologous recombination. Expression of the transposase from this integrated gene could provide sufficient transposase for excision and re-excision of the transposon, thus increasing the risk of genotoxicity. Embodiments of the invention are directed to methods that avoid this potentially deleterious effect on the host cell genome by delivering either an mRNA encoding the transposase, or by delivering the transposase polypeptide itself. Transgene insertion therefore only takes place until the transposase mRNA and/or proteins become degraded by cellular housekeeping enzymes.

Although gene therapy has been promoted optimistically for over a decade, the formidable technical problems and safety concerns have yet to be successfully addressed. The clinical trials to date have used inactivated viruses as vectors to shuttle transgenes into patients cells, but these viruses are partly to blame for the devastating outcomes of such trials, which have led to the onset of Leukemia and even the death in some patients. There have been several attempts to circumvent the problems associated with viral gene therapy, such as one approach using “gutless” viral vectors for delivery of transposons into patients' cells. However, transposons integrate into random sites in the genome, leading to insertional mutations. Such mutations are likely to result in the onset of genetic alterations that can trigger disease, as is the case with Leukemia often observed following use of Lentiviral vectors. Other vectors undergoing testing for gene therapy utilize a bacterial site-specific recombination system called a “bacteriophage” integrase. This vector has the ability to insert large DNA fragments into cultured cells in a pseudo-site-specific manner, but is relatively ineffective in animals. The pseudo-site-specificity also introduces the risk of cancer development via the deactivation of cancer supressor genes which can contain the pseudo-sites for insertion preferred by the bacteriophage.

Correct gene expression is part of an individual's development and well being, as aberrant gene expression leads to disorders and genetic disease. Some aspects of the invention are directed to methods of reducing the risks associated with many currently-proposed methods of gene therapy by utilizing chimeric transposon technology (CTT). Embodiments of the invention encompass the use of chimeric piggyBac transposases including the DNA binding domains of transcription factors in gene therapy procedures. Such domains recognize and bind to specific DNA sequences within or near a particular gene sequence. Some classes of transcription factors are characterized by their zinc binding capacity and are known as zinc finger DNA binding proteins (ZFPs). The DNA recognition and binding function of ZFPs can be used to target a variety of functional domains in a gene-specific location. The recognition domain of ZFPs is composed of two or more zinc fingers; each finger recognizes and binds to a three base pair sequence of DNA and multiple fingers can be linked together to more precisely recognize longer stretches of DNA. Embodiments of the invention encompass chimeric transposases with engineered ZPFs whose DNA-interacting amino acid residues can be modified to recognize specific DNA sequences in variety of different genes. PiggyBac-encoding vectors containing CTT elements for gene therapy trials are described herein. The use of vectors including chimeric piggyBac, Sleeping Beauty, or Tol2 transposases in transgenesis of cultured human cells is described in Example 6.

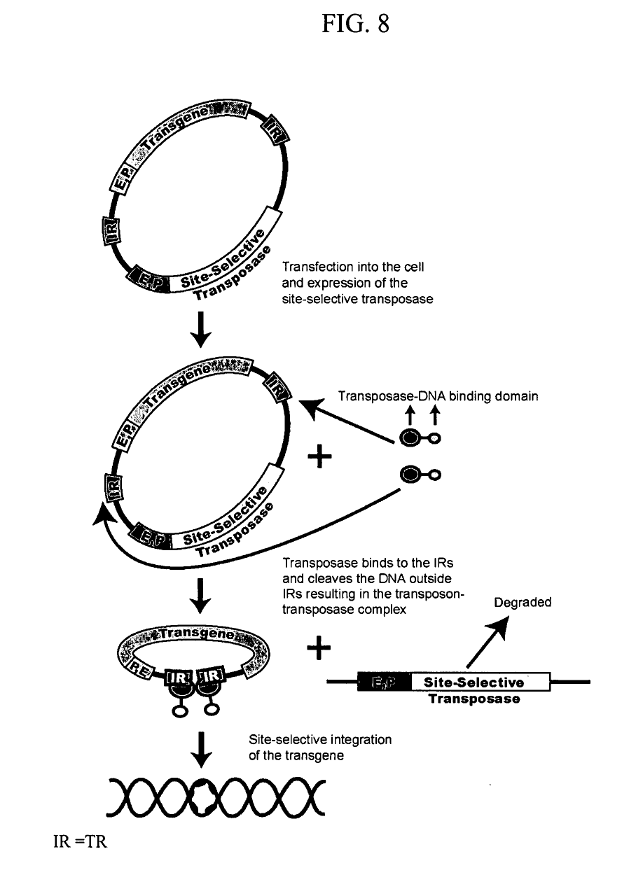

Traditional methods of transgenesis result in gene insertion at random locations within the large genome of higher organisms, resulting in loss of efficiency, unpredictable results and unintended genetic consequences. CTT can target a specified, unique site within the genome, eliminating these disadvantages. Unlike other methods of targeted gene insertion, the site targeted by CTT can be “programmed” at will by modifying the amino acid contacts of ZFPs for DNA as described above. The insertion of a ZFP sequence at the 5′-end of the piggyBac gene does not interfere with the activity of the protein produced, and such ZFPs can demonstrate target specificity. In addition to gene therapy attempts in whole animals with such vectors, embodiments of the invention are directed to methods of determining their effectiveness in inserting genes at specific sites using intracytoplasmic sperm injection (ICSI) of the vectors into mouse oocytes. The short gestation period of twenty-one days in the mouse facilitates interpretable results for the insertion of transgenes by such CTT vectors, which additionally contain a kanamycin resistance gene for plasmid rescue experiments within the transposon. The diagram in FIG. 8 highlights the main structural components of this approach. Once inside the fertilized oocyte, the construct finds its way into the newly formed zygotic nucleus. The transcriptional machinery within the nucleus transcribes the transpose gene (piggyBac and others) with its nuclear localization signal (NLS). The translational machinery of the zygote synthesizes the protein, which by virtue of its NLS makes its way into the nucleus. There, the newly synthesized transposase binds to the terminal repeats (TR) which flank the transposon. The catalytic domain (large circle) of the transposase recognizes the TRs and prepares the DNA between them for insertion. The ZFP DNA binding domain (small circle) guides the insertion complex to a unique site on the DNA of the host genome and the transposase protein performs the insertion of the transposon. Information gained during animal experiments can transfer to human gene therapy trials and help in the development of an alternative technique to the controversial retroviral methods currently used in human gene therapy.

Plasmids encoding piggyBac transposase chimeric for the ZFPs can be injected into mouse oocytes during ICSI and recovered genomic DNA from founder animals are assayed for gene insertion. This is achieved by selecting circularized genomic DNA constructs which act like plasmids by virtue of the activity of the kanamycin antibiotic gene present in the rescue plasmid. This allows the selection of bacteria cells that have incorporated the circularized DNA during transformation. Bacteria that survive selection in kanamycin medium have the gene region of interest incorporated into them. This circularized DNA can be recovered like a plasmid and the region of interest containing the transposon amplified by PCR with primers specific to the transposon. Such PCR amplified regions are then sequenced and the site of integration for the transgene recognized. Embodiments of the invention with the single donor and helper pMMK-1 and pMMk-2 plasmids make it possible to conduct the CTT gene therapy approaches described above.

Further embodiments of the invention relate to methods of generating a transgenic animal including the steps of: contacting with a round spermatid a nucleic acid including a transposable exogenous nucleotide sequence and a nucleotide sequence encoding a transposase on the same nucleic acid; and introducing the nucleic acid contacted with the spermatid into an artificially activated oocyte to form a transgenic embryo, whereby the transposase is expressed in the embryo and catalyzes integration of the transposable exogenous nucleotide sequence into the genome of the embryo; and implanting the transgenic embryo into a suitable surrogate mother of the same species under conditions favoring the development of the transgenic embryo into a transgenic offspring.

Methods of generating a transgenic animal using transposase enzymes has previously been described. See U.S. Provisional Application No. 60/840,780, filed on Aug. 28, 2006, entitled CELL AND ANIMAL TRANSGENESIS WITH SINGLE PLASMID TRANSPOSASE (HELPER) AND TRANSPOSON (DONOR) CONSTRUCTS; U.S. Provisional Application No. 60/840,833, filed Aug. 28, 2006, entitled TRANSGENESIS-READY MICE CONTAINING TRANSPOSASE ENZYME GENES IN THEIR GENOME, DRIVEN BY OOCYTE-SPECIFIC DEVELOPMENTAL PROMOTER; U.S. Provisional Application No. 60/859,652, filed Nov. 17, 2006, entitled RNA AS A SOURCE OF TRANSPOSASE OR THE PROTEIN TRANSPOSASE FOR PIGGYBAC MEDIATED GENE INSERTION AND EXPRESSION; and U.S. application Ser. No. 11/127,685, filed May 11, 2005, entitled ACTIVE TRANSGENESIS WITH USE OF TRANSPOSOME DURING ICSI WITH NON-FREEZE-THAWED FRESH SPERM. Each of the applications, including all methods, figures, and compositions, is incorporated herein by reference in its entirety.

Having described embodiments of the invention in detail, it will be apparent that modifications, variations, and equivalent embodiments are possible without departing the scope of the invention defined in the appended claims. Furthermore, it should be appreciated that all examples in the present disclosure are provided as non-limiting examples.

### EXAMPLES

The following non-limiting examples are provided to further illustrate the present invention. It should be appreciated by those of skill in the art that the techniques disclosed in the examples that follow represent approaches the inventors have found function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Example 1

### Generation of Transgenic Mice Using a Single Plasmid Encoding PiggyBac Transposase and Carrying a PiggyBac Transposon Encoding an EGFP Reporter Protein

Ten microliters of 200 nanogram/microliter plasmid pMMK-2 (FIG. 2) is mixed with 10 microliters of fresh swim-up sperm solution. Each sperm head that has its tail removed in the mixed solution is individually microinjected into a metaphase II (MII) arrested matured mouse oocyte (intra-cytoplasmic sperm injection, ICSI). Alternatively, plasmid DNA alone is injected into a fertilized oocyte (pronuclear microinjection). Two-cell embryos are transferred into the oviducts of pseudopregnant females which are mated with vasectomized males the night before. The females are allowed to give birth to their own young and the newborn pups are examined for EGFP expression in their skin by epifluorescence (FIG. 9).

Table 1 indicates the rates of transgenic mice generation (percentage of transgenic animals born for every oocyte injected) using the method of microinjection and concentration of plasmid pMMK-2 shown.

TABLE 1
Transgenesis rates in mice after microinjection of plasmid pMMK-2 into mouse oocytes.

| | | | | | | |
|---|---|---|---|---|---|---|
| Number of | | | | | | |
| oocytes | | DNA | Embryos | | | % Transgenic |
| injected | Method | concentration | transferred | Animals | Animals | (animals born) |
| (repetitions) | (Promoter) | (volume injected) | (Surrogate #) | born | transgenic | [oocytes injected] |

| | | | | | | | |
|---|---|---|---|---|---|---|---|
| 91 (1) | Pronuclear | 1 ng/μL (15 μm) | 61 (2) | 11 | 0 | — | — |
| | (CAG) | | | | | | |
| 159 (1) | Pronuclear | 10 ng/μL (15 μm) | 122 (3) | 15 | 4^{C} | (26.6) | [2.5] |
| | (CAG) | | | | | | |
| 74 (2) | ICSI (CMV) | 100 ng/μL (15 μm) | 63 (5) | 38 | 1^{C} | (2.6) | [1.4] |
| 104 (3) | ICSI (CAG) | 100 ng/μL (15 μm) | 82 (5) | 49 | 6^{C} | (12.2) | [5.8] |
| 36 (1) | ICSI (CAG) | 100 ng/μL (50 μm) | 27 (2) | 13 | 4^{C} | (31.0) | [11.1] |
| 33 (2) | ICSI (CAG) | 100 ng/μL (100 μm) | 29 (2) | 9 | 4^{C} | (44.4) | [12.1] |
| 79 (2) | ICSI (CAG) | 150 ng/μL (100 μm) | 69 (4) | 26 | 18^{C} | (69.2) | [22.8] |
| 72 (2) | ICSI (Donor | 150 ng/μL (100 μm) | 57 (3) | 19 | 0 | 0 | 0 |
| | only) | | | | | | |

### Example 2

### Single-Plasmid Transgenesis in Human HEK293 Cells Using PMMK-1

To determine the efficiency of transgenesis in human cells using a single plasmid carrying both a piggyBac transposon and the gene for piggyBac transposase, HEK293 cells are transfected with the plasmid pMMK-1 (FIG. 1), which carries a transposon containing the hygromycin resistance gene. HEK293 cells are maintained in MEM alpha medium (Hyclone) containing 5% FBS (Hyclone). Cells at 80% confluence are harvested, and 1×10⁵ cells are seeded into individual wells of 24-well plates 18 hours before transfection. A total of 400 ng of DNA are used for each transfection with FuGENE 6 (Roche). One-tenth of the transfected cells is transferred to 100 mm plates followed by hygromycin selection for 14 days. The concentration of hygromycin B used in HEK293 cells is 100 μg per milliliter. To count the number of clones, cell colonies are fixed with phosphate-buffered saline (PBS) containing 4% paraformaldehyde for 10 minutes and then stained with 0.2% methylene blue for 1 hr. After 14 days of hygromycin selection, only colonies larger than 0.5 mm in diameter are counted. FIG. 3 shows the number of hygromycin resistant colonies counted after transfection with pMMK-1 as compared with the number seen after transfection with a control plasmid lacking the piggyBac coding sequence. Transgenesis mediated by piggyBac encoded on pMMK-1 is 9.4-fold more frequent than random insertions in cells transfected with the control plasmid.

### Example 3

### PiggyBac Exhibits Greater Transposition Activity in Mammalian Cells than SB11, Tol2, and Mos1

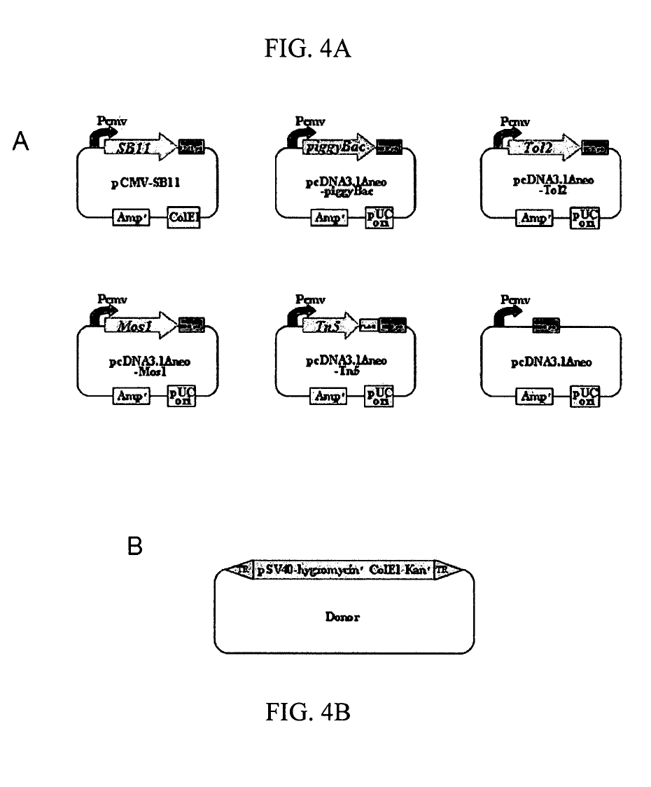

Since different transposon systems have been independently developed and tested in different laboratories, it is difficult to draw conclusions regarding their relative efficiency only on the basis of published literature. A direct comparison of transposition activity of various transposon systems identifies the most promising transposon(s). Mos1, SB11, Tol2, and piggyBac transposon systems can be constructed using a two-component system (FIG. 4): a helper plasmid containing the transposase driven by the cytomegalovirus (CMV) promoter (FIG. 4A) and a donor plasmid with the terminal repeats bearing a cassette with hygromycin resistance and kanamycin resistance genes to facilitate selection in eukaryotes and prokaryotes, respectively, and a ColE1 replication origin for plasmid propagation in bacteria (FIG. 4B). Additionally, the efficiency of chromosomal integration can vary among the transposons depending upon chromatin organization and/or host factors. Therefore, transposition activity of each of the four transposon systems is determined in four mammalian cell lines: HeLa (human cervical carcinoma), HEK293 (human embryonic kidney cell), H1299 (human lung carcinoma), and CHO (Chinese hamster ovarian carcinoma).

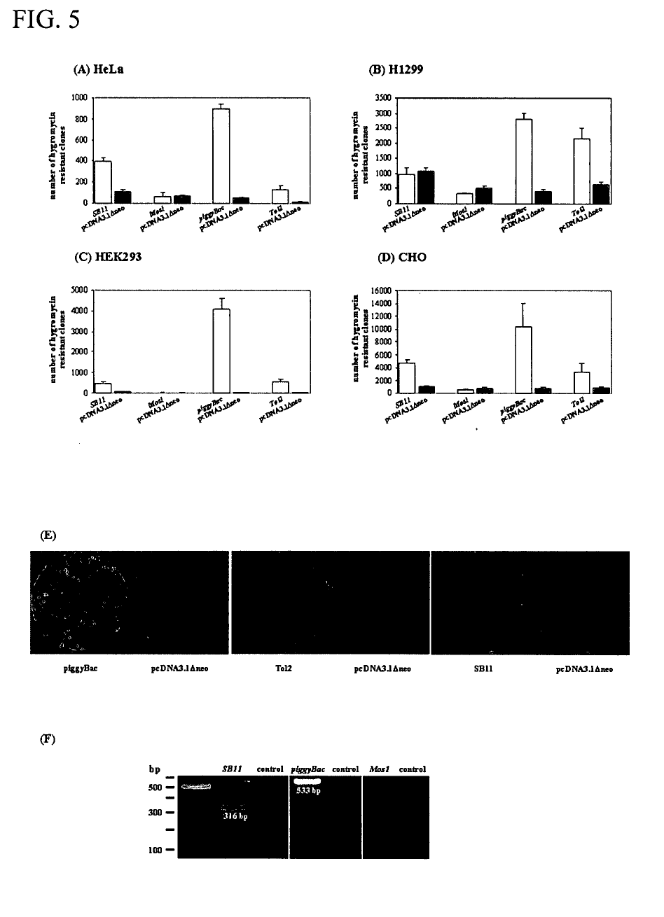

To assess efficiency of transgenesis, cells at 80% confluence are harvested, and 1×10⁵ cells are seeded into individual wells of 24-well plates 18 hours before transfection. A total of 400 ng of DNA is used for each transfection reaction with FuGENE 6 (Roche). For each cell line, one-tenth of the transfected cells is transferred to 100 mm plates followed by hygromycin selection for 14 days. The concentration of hygromycin B used in HeLa, HEK293, H1299, and CHO cells is 200, 100, 400, and 400 μg per milliliter, respectively. To count the clones, cells are fixed with PBS containing 4% paraformaldehyde for 10 min and then stained with 0.2% methylene blue for 1 hr. After 14 days of hygromycin selection, cell colonies are counted. Because colonies smaller than 0.5 mm in diameter often fail to be subcloned in the presence of hygromycin, only colonies larger than 0.5 mm in diameter are counted. As shown in FIG. 5 (A-E), piggyBac and Tol2 possess activity in all cell lines tested. SB11 displays slight transposition activity in CHO, HeLa, and HEK293 cells, while it is inactive in H1299 cells. No transposition activity is detected with Mos1 in the four cell lines used (FIG. 5A-E).

As indicated in FIG. 5A-D, the transposition activity of piggyBac, Tol2, and SB11, varies in different cells. For example, ˜1000 hygromycin-resistant colonies are detected with both the control plasmids and SB11-expressing plasmid in H1299 cells (FIG. 5B), suggesting a lack of transposition activity of SB11 in this cell line. However, in HEK293 cells, ˜500 hygromycin-resistant colonies are detected in the presence of SB11 transposase, which represents an 8-fold increase over cells transfected with control plasmid (FIG. 5C). Two parameters, relative fold and percentage of transposition, are thus used to assess the transposition activity of the different transposon systems. The relative fold is obtained by dividing the number of hygromycin-resistant colonies detected in cells transfected by donor plus helper by the colony number that results from random integration (as in transfection experiments with control plasmid not encoding a transposase). The percentage of transposition, hereafter referred to as transposition rate, is calculated by subtracting the number of hygromycin-resistant colonies detected in cells transfected with control plasmid from the number of resistant colonies observed in cell transfected with a transposase-encoding plasmid, then dividing by 1×10⁵ (the number of cells originally seeded before transfection), and finally multiplying by 100 to convert to percentage. The transposition rate represented here, however, is not normalized by the transfection efficiency in various cell lines. As summarized in Table 2, the relative fold ranges for the three transposons in different cell lines are as follows: (1) SB11 from 1 [equal to control] in H1299 to 8.1 in HEK293, (2) piggyBac from 5.7 in H1299 to 114 in HEK293, and (3) Tol2 from 3.3 in CHO to 93.9 in HEK293. The transposition rate ranges are: (1) SB11 from 0% in H1299 to 2.9% in CHO, (2) piggyBac from 0.7% in HeLa to 7.0% in CHO, and (3) Tol2 from 0.08% in HeLa to 1.8% in CHO. Once again, piggyBac displays the highest transposition activity among the three active transposon systems tested, as judged by both the transposition rate and relative fold. The transposition rate of Tol2 is higher than SB11 in H1299 and HEK293 but not in CHO and HeLa cells. Owing to the relatively high integration rate of the SB11 control, the relative fold seen in all four cell lines for Tol2 is higher than that of SB11.

TABLE 2
Summary of the transposition efficiency of SB11, piggyBac, and Tol2 transposon systems.

| | SB11 | | piggyBac | | Tol2 | |
|---|---|---|---|---|---|---|
| | Relative | Percentage of | Relative | Percentage of | Relative | Percentage of |
| Cells | fold | transposition | fold | transposition | fold | transposition |
| CHO | 2.9 ± 1.6 | 2.3 ± 1.5 | 9.8 ± 6.4 | 7.0 ± 3.9 | 3.3 ± 1.7 | 1.8 ± 1.2 |
| HeLa | 2.7 ± 1.0 | 0.2 ± 0.1 | 14.5 ± 3.5 | 0.7 ± 0.2 | 5.4 ± 3.2 | 0.08 ± 0.03 |
| H1299 | 1.0 ± 0.16 | 0 | 5.7 ± 1.3 | 2.2 ± 0.2 | 4.1 ± 0.8 | 1.4 ± 0.3 |
| HEK293 | 8.1 ± 2.1 | 0.4 ± 0.1 | 114.6 ± 81.6 | 2.8 ± 1.4 | 93.9 ± 75.8 | 0.9 ± 0.3 |

Relative fold values indicate the relative fold of hygromycin-resistant clones as compared with controls (n = 6).
Percentage of transposition values indicate the percentage of true transposition from 1 × 105 cells seeded.

The type of DNA transposition described herein involves a two-step action: (1) excision of the transposable element from the donor plasmid, and (2) integration of the excised fragment into its DNA target. Therefore, the numbers of hygromycin-resistant colonies are the result of both excision and integration events. Although no activity is detected in cells transfected with Mos1, it is still possible that successful excision occurs but that integration does not. To exclude this possibility, a plasmid-based excision assay is performed using the polymerase chain reaction (PCR). As a consequence of excision, a short version of the donor plasmid should be produced.

To perform this assay, one million HEK293 cells are seeded onto 60 mm plates 18 hours before transfection. One microgram each of donor and helper plasmid is transfected into the cells. Plasmids are recovered using the Hirt method 72 hours after transfection. Ziegler, K., et al. J. Virol. Methods [2004] 122:123. Plasmids isolated are used as templates for nested PCR using primers listed below to detect the presence of donor plasmids that undergo excision: piggyBac first round, 5Bac-1(TCGCCATTCAGGCTGCGC)/3Bac-1(TGTTCGGGTTGCTGATGC); piggyBac second round, 5Bac-2(CCTCTTCGCTATTACGCC)/3Bac-2(TGACCATCCGGAACTGTG); Sleeping Beauty first round, F1-ex (CCAAACTGGAACAACACTCAACCCTATCTC)/o-lac-R(GTCAGTGAGCGAGGAAGCGGAAGAG); Sleeping Beauty second round, KJC031(CGATTAAGTTGGGTAACGCCAGGGTTT)/i-lac-R(AGCTCACTCATTAGGCACCCCAGGC); Mos1 first round, 5mos-1(TCCATTGCGCATCGTTGC)/3mos-1 (AGTACTAGTTCGAACGCG); Mos1 second round, 5mos-2 (ACAGCGTTGTTCCACTGG)/3mos-2 (AAGCTGCATCAGCTTCAG).

No excision-dependent PCR product is detected in cells transfected with donor and helper plasmids for Mos1, whereas excision-dependent PCR products with sizes of 533 by for SB11 or 316 by for piggyBac are detected (FIG. 5F). SB11 and piggyBac are therefore able to both excise and integrate their respective transposons, while Mos1 is unable to do either.

### Example 4

### PiggyBac Exhibits Greater Transposition Activity in Mammalian Cells than Tol2 and SB11 in Chromosomal Integration Assay Testing Varying Amounts of Helper Plasmid

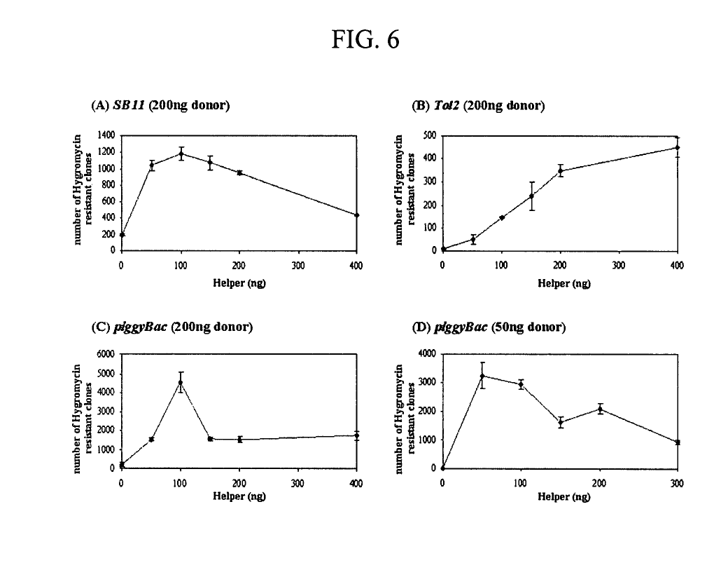

To confirm that piggyBac is more efficient than Tol2 and SB11 transposases, a chromosomal integration assay is performed by transfecting HEK293 with a fixed amount of donor plasmid (200 ng) plus varying amounts of helper plasmid encoding either piggyBac, Tol2, and SB11. Plasmid pcDNA3.1Δneo (FIG. 4B) is used to normalize the total amount of DNA introduced into the cells. As shown in FIG. 6A-C, the lowest number of hygromycin-resistant colonies for piggyBac is approximately 1500, which is significantly higher than the highest number of resistant colonies observed for either Tol2 (490) or SB11 (1180). Furthermore, piggyBac achieves its highest transposition activity (4535) when 200 ng of donor and 100 ng of helper plasmids are introduced into cells. Therefore, piggyBac consistently demonstrates the highest transposition activity of the four transposases tested in mammalian cells in this study.

### Example 5

### PiggyBac Transposition Declines as Helper Levels Increase

Transposition efficiency depends on the availability of transposon (donor) and transposase (helper) in cells. It was shown elsewhere that, over a certain threshold, SB11 transposition declines with increasing transposase, a phenomenon known as overproduction inhibition. Lohe, A et al., Mol Biol Evol [1996] 13:549. Conversely, Tol2 transposition was directly proportional to the levels of transposase and did not appear to exhibit overproduction inhibition. Kawakami, K et al., Genetics [2004] 166:895. Overproduction inhibition for SB11 is also observed, while Tol2 transposition is directly proportional to the amount of transposase DNA (FIG. 6A, B). Zayed, H., et al., Mol Ther [2004] 9:292. Like SB11, piggyBac also shows peak activity at a ratio of 2 to 1 (donor to helper). However, unlike SB11, which demonstrates a gradual reduction of activity above this ratio, the activity of piggyBac declines rapidly (FIG. 6C). These findings suggest that piggyBac exhibits overproduction inhibition.

To further address this issue, a chromosomal integration assay is performed for piggyBac using 50 ng of donor with increasing amounts of helper ranging from 50 to 300 ng. For each transfection, pcDNA3.1Δneo is again used to normalize the total amount of DNA introduced into the cells. As seen in FIG. 6D, increasing the ratio of helper to donor plasmid beyond the 2:1 ratio producing optimum transgenesis results in a gradual reduction in transposition. These data further tend to indicate that overproduction inhibition occurs with piggyBac.

### Example 6

### Activity of a GAL4-PiggyBac Chimeric Transposase is Similar to that of the Wild Type Transposase

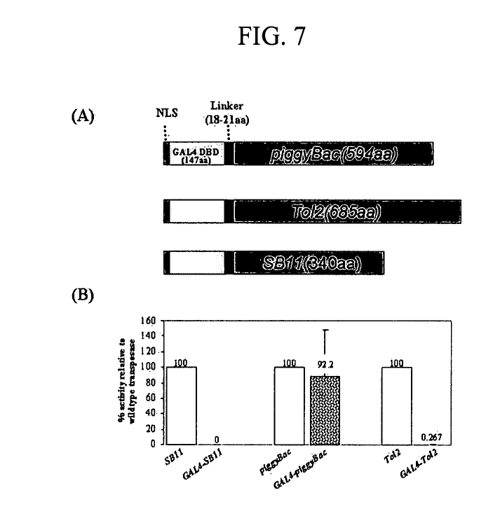

Directing transgene integration to a unique and safe site on the host chromosome can overcome the hazards of insertional mutagenesis that can result with integrating vectors currently in use. A transposon-based gene delivery system preferably features a custom-engineered transposase with high integration activity and target specificity. Targeting transposon integration to specific DNA sites using chimeric transposases engineered with a DNA binding domain (DBD) has been demonstrated in mosquito embryos containing a plasmid including a unique site recognized by a GAL4 DNA binding domain fused to a transposase. Maragathavally, K J, FASEB J. [2006] 20:1880. Such modifications can render a transposase inactive, however, as observed for variants of SB11 engineered for target specificity, which have dramatically reduced transposition activity. Wilson, M H FEBS Lett [2005] 579:6205. Therefore, the potential for modifications of SB11, Tol2, and piggyBac transposases can be assessed by testing their activity when fused to a GAL4 DNA binding domain. The transposition activities of each of these transposases, upon addition of an N-terminal GAL4 DBD (FIG. 7A), can be determined using a chromosome integration assay in HEK293 cells. GAL4-piggyBac transposase demonstrates transposition activity similar to that of wild-type piggyBac, while GAL4-Tol2 and GAL4-SB11 transposases possess negligible activity, even though GAL4-SB11 protein is detected by Western blot using a monoclonal antibody (FIG. 7B).

PiggyBac inserts into the tetranucleotide site TTAA, which is duplicated upon insertion. Ding, S, Cell [2005] 122:473; Tosi L R, Nucleic Acids Res [2000] 28:784. To test whether fusion of GAL4 to the N-terminus of piggyBac transposase alters its preference for TTAA sites, plasmid rescue experiments can be performed to retrieve the sequence information of the target sites using genomic DNAs isolated from individual hygromycin-resistant CHO cell clones. Individual clones are isolated and allowed to grow to confluence in a 100 mm plate. Genomic DNA is isolated using a DNeasy Tissue kit according to the manufacturer's protocol (Qiagen). Five micrograms of genomic DNA is subjected to Xhol digestion followed by ligation into a plasmid containing a bacterial origin of replication and an antibiotic resistance gene. The ligation reactions are transformed into E. coli DH10B cells. Plasmids rescued from transformants are subjected to DNA sequencing to retrieve the genomic sequence flanking the insertion site. Six independent genomic sequences are recovered from four drug-resistant clones. As shown in Table 3, all of these sequences contain genomic DNA with the signature TTAA sequence at the integration site. This experiment demonstrates that the chromosomal integrations observed in cells transfected with GAL4-piggyBac are mediated by a true transposition event with the same insertion preference for TTAA sites. Thus, neither the mechanism of transposon insertion by piggyBac transposase, nor its high level of activity appear, appear to be effected by fusion to a site-selective GAL4 DBD.

TABLE 3
Analysis of chromosomal insertion site selection by
GAL4-piggyBac transposase in CHO cells

| Independent | Donor plasmid | Chromosomal | Flanking |
|---|---|---|---|
| Isolated Clones | terminal repeat | Insertion Site | Chromosomal Sequence |
| G8-2 | ^{5′}TGATTATCTTTCTAGGG | TTAA | GCTCGGGCCGGCCGCGTCGCCGCTTC^{3′} |
| G25-2 | TGATTATCTTTCTAGGG | TTAA | CAATCAATAAGATAAACATACACAGA |
| G25-3 | TGATTATCTTTCTAGGG | TTAA | CACCACATTTAACTTGCTCTTTGATA |
| G28-1 | TGATTATCTTTCTAGGG | TTAA | TAGAGTGCTGAGATTTGGGACATTGC |
| G29-1 | TGATTATCTTTCTAGGG | TTAA | GGCGTTGGTGGCACACAACTTTAAGT |
| G34-2 | TGATTATCTTTCTAGGG | TTAA | TAAGACAATGTATGACTTTGTCCCAT |

### Example 7

### Construction of Plasmids PMMK-1 and PMMK-2

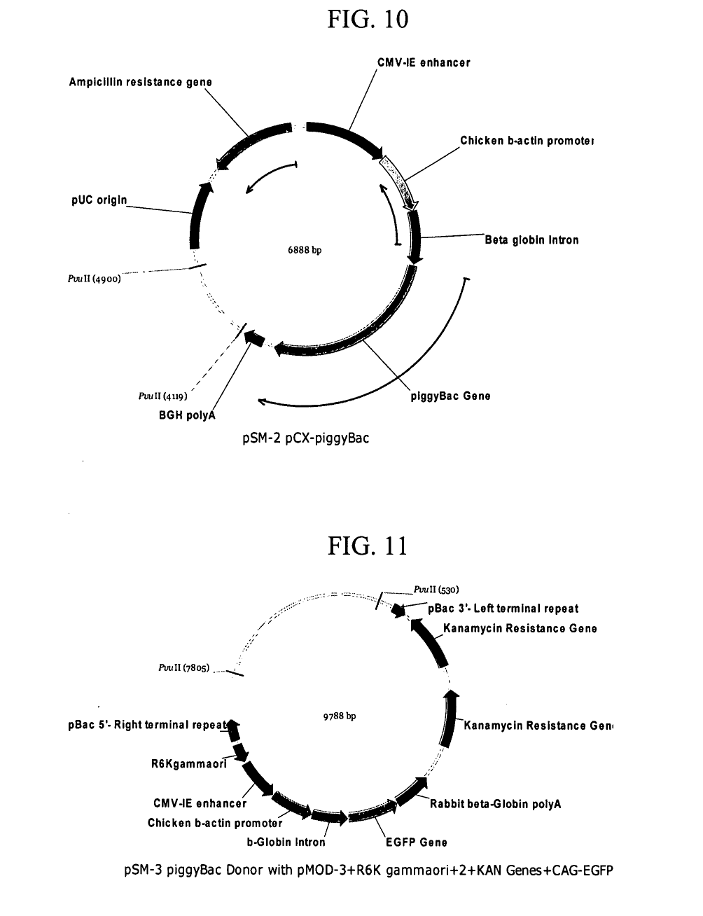

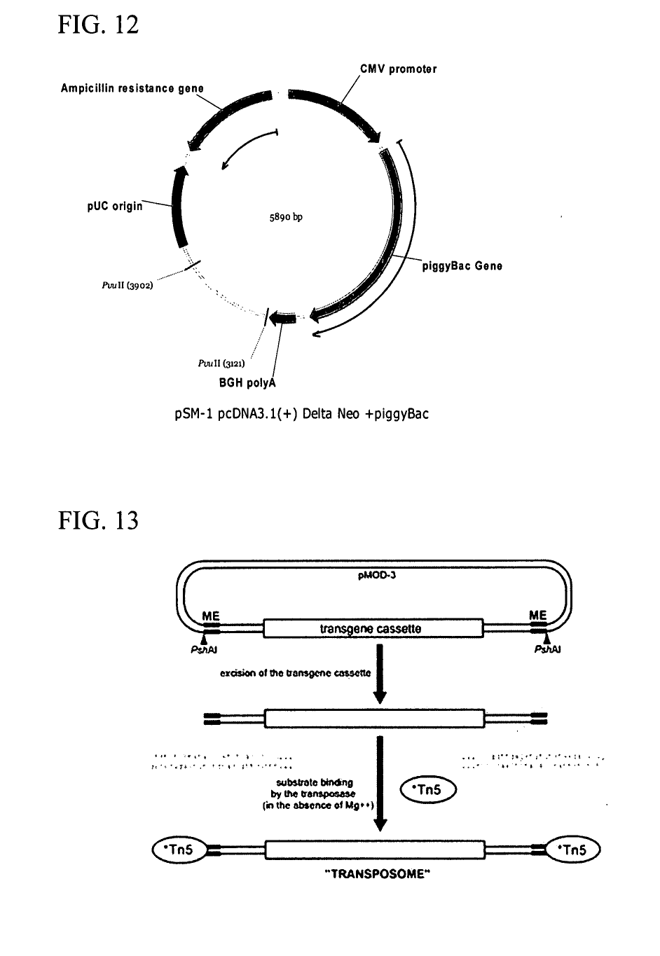

To generate plasmids containing both a piggyBac transposon and the gene for piggyBac transposase, 2 μg of plasmid pSM-2 (FIG. 10) containing the piggyBac transposase gene driven by the CAG promoter (a combination of chicken beta-actin promoter and cytomegalovirus immediate-early enhancer), is restriction digested with the enzyme PvuII and phosphatased. Two μg of piggyBac transposon-containing donor plasmid pSM-3 (FIG. 11), containing the CAG promoter-driven gene for EGFP and two R6K gamma on activated kanamycin resistance genes, all flanked by the 3′- and 5′-end terminal repeats (TR) recognized by piggyBac transposase, is also digested with the restriction enzyme PvuII (FIG. 11). The larger 6107 by pSM-2 and 7275 by pSM-3 fragments containing the helper and donor portions of their respective plasmids are gel purified in 1% agarose gels, and purified together from excised gel bands using a Zymo-Clean column. To 8 microliters of the flow through, 1 microliter of 10×T4 DNA ligase buffer and 1 microliter of T4 DNA ligase enzyme are added, and the mixture incubated overnight at 16° C. Three microliters of the ligation reaction is transformed into 200 microliters of DH5α competent bacterial cells. Fifty microliters of transformed cells are plated on LB agar containing 100 micrograms/milliliter ampicillin and incubated overnight at 37° C. Cloned ligation products are purified, the two kanamycin genes removed with BamHI restriction digestion, and a cassette for pSV40-hygromacin and ColE1 kanamycin is inserted in their place to give rise to plasmid pMMK-1 to be used for HEK293 cell transfection (FIG. 1). This process (ligation of PvuII-digested plasmids) is also performed for plasmid pSM-1, containing the piggyBac transposase gene driven by the CMV promoter (FIG. 12), and piggyBac donor plasmid pSM-3 (FIG. 11) to give rise to the transfection plasmid pMMK-2 (FIG. 2).

### Example 8

### In-Vitro Transcribed mRNA Encoding PiggyBac Transposase Mediates Transposition in Human Cells

As an alternative to introducing the piggyBac transposase gene into cells on the same plasmid as the piggyBac transposon (pMMK-1 and pMMK-2), the piggyBac transposase gene can also be encoded on an mRNA that is co-introduced into cells with a donor plasmid not encoding the transposase. In this case, expression of the transposase is not delayed by the gene's transcription, and genomic integration of the transposon can have a greater chance of occurring before the embryo's first division, thus producing non-mosaic offspring with an integrated copy of the transgene in each of its cells. Capped RNA transcripts are generated in vitro from a plasmid template encoding piggyBac transposase using T3 RNA polymerase (Riboprobe in vitro Transcription System by Promega). This system produces 7-methylguanosine (m₇G)-capped RNAs encoding the piggyBac transposase stabilized with 5′ and 3′ untranslated sequences from the Xenopus laevis β-globin gene. Following transcription, the RNA is treated with DNase1 to digest the DNA template. RNA is purified by lithium chloride precipitation, washed twice with 70% ethanol, and resuspended.

To attempt this method of transgenesis first in tissue culture cells, 5×10⁵ cells (such as HeLa, CHO, HEK293, H1299, HT1080) are seeded into 6 cm culture plates. Cells are co-transfected the following day with the donor plasmid (FIG. 4) and in vitro-transcribed RNA encoding transposase (500 ng of each) with SuperFect reagent (Qiagen). Forty-eight hours later, 50,000 cells are plated into 100 mm dishes containing complete growth medium supplemented with hygromycin. After 14 days of selection, hygromycin resistant colonies are fixed and stained with 70% methanol/1% crystal violet to determine the frequency of hygromycin resistant colony formation.

### Example 9

### Transgenesis with Transposase Polypeptide Coinjected into Mouse Embryos with Tranposon Donor Plasmid

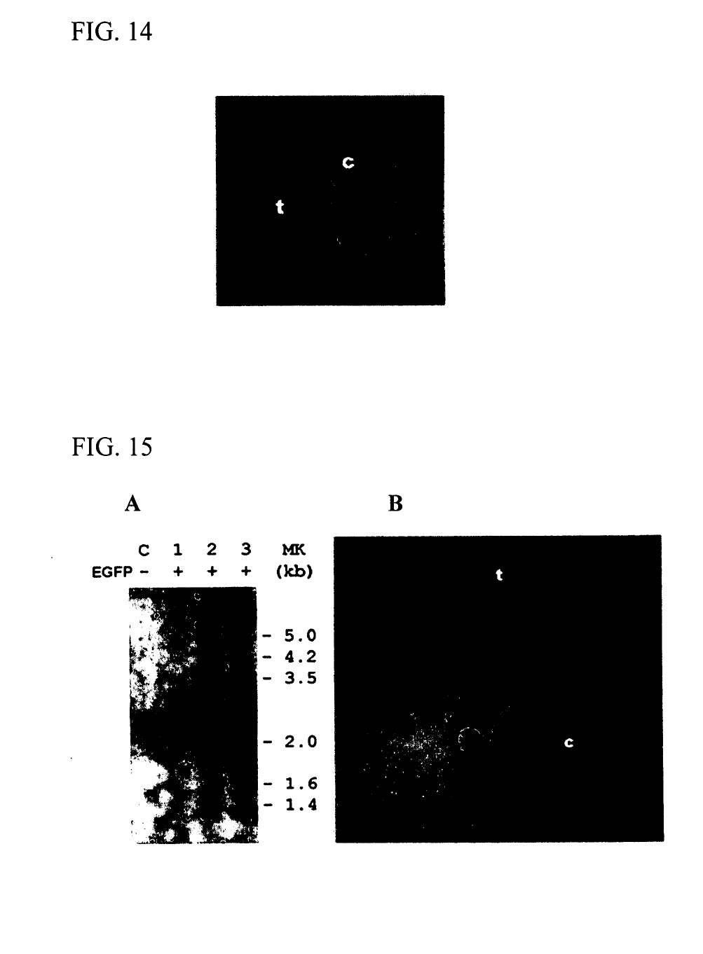

Some embodiments of the present invention relate to methods of generating a transgenic animal or cell using a piggyBac transposase polypeptide coinjected or cotransfected with a transposon donor plasmid. A similar integrating enzyme, the bacterial transposase Tn5, used in this manner efficiently generates mice embryos carrying an EGFP transgene. In this study, intracytoplasmic sperm injection (ICSI) is employed, as well as other microinjection-based methods for transgenesis of hybrid (B6D2F1) and inbred (C57BL/6) strains of mice. Delivery and integration of the EGFP-coding transgene into the mouse embryo genome is carried out with the help of a hyperactive mutant of the Tn5 transposase protein designated *Tn5p (Reznikoff W S, Mol Microbiol 47: 1199-1206 (2003); Naumann T A and Reznikoff W S, J Biol Chem 277: 17623-17629 (2002)) (FIG. 13). The *Tn5p:DNA complexes or “transposomes” shown, resembling natural Tn5 transposition intermediates, are formed by allowing the purified transposase to bind to its terminal repeat (ME) recognition sequences in the absence of Mg²⁺ ions. Freshly isolated sperm heads are individually co-injected into mouse metaphase II (MII) oocytes with either naked dsDNA alone, as described in previous ICSI transgenesis studies (ICSI-Tr) (Perry A C, et al., Science 284: 1180-1183 (1999); Perry A C, et al., Nat Biotechnol 19: 1071-1073 (2001)), or as a *Tn5p:DNA complex (TN:ICSI). The DNA fragment used to construct the transposome contains an EGFP gene driven by a CAG promoter (Ikawa M, et al., FEBS Lett 375: 125-128 (1995)) similar to pMMK-1 and pMMK-2 (FIGS. 1 and 2). Two-cell embryos are then transferred into oviducts of surrogate females and allowed to develop to full term. All of the resulting F0 transgenic progeny are recognized for transgene expression by epifluorescence of EGFP (FIG. 14, t). Control progeny (FIG. 14, c) do not exhibit epifluorescence. In a control experiment (ICSI using only transposon DNA), only one pup is germline transgenic and shows weak mosaicism.

TABLE 4
Summary of TN:ICSI, TN:ROSI, pronuclear and cytoplasmic injection experiments with Tn5 transposomes

| | | | Number | | | | | | | | | | |
|---|---|---|---|---|---|---|---|---|---|---|---|---|---|
| | | Male | of | Number | | | | Number of | | | Efficiency | | |
| | Method | germ | oocytes | of | Number of | | | transferred | | Live | | | % |
| | used | cell | used | surviving | embryos reach | | | embryos | | offspring | | Germine: | oo- |
| Authors | (strain) | treatment | (Repetitions) | oocytes | 2 + C^{#} | m | b | (Recipients) | | (% oocytes) | Transgenic: | Transgenic | cytes |
| Perry et al., | ICSI | Freeze- | 97 (1) | — | — | 53 | — | 53 | (3) | 12 (12.4) | 2 | 2 | 2.0 |
| 1999 | (Hybrid) | thaw | | | | | | | | | | | |
| Perry et al., | ICSI | Freeze- | — | (213) | 45 | 164 | 4 | 179 | (12) | 14 (6.6)^{a} | 6 | 5 | (2.8)^{a} |
| 2001 | (Hybrid) | thaw | | | | | | | | | | | |
| Moreira et | ICSI | Freeze- | 219 (6) | 195 | 152 | — | — | 163^{b} | (8) | 22 (10.0) | 10 | — | 4.6 |
| al., | (Outbred) | thaw | | | | | | | | | | | |
| 2004 | | | | | | | | | | | | | |
| Moreira et | ICSI | Freeze- | 367 (6) | 252 | 201 | — | — | 218^{c} | (13) | 34 (9.2) | 12 | — | 3.3 |
| al., | YAC | thaw | | | | | | | | | | | |
| 2004 | (Outbred) | | | | | | | | | | | | |
| Lois et al., | Lentiviral | — | 270 (2) | 231 | — | — | — | 197 | (—) | 73 (27.0) | 63 | — | 23.3 |
| 2002 | (Hybrid) | | | | | | | | | | | | |
| Nakanishi | Pronuclear | — | 4739 (—) | — | — | — | — | — | (182) | 626 (13.2) | 150 | — | 3.2 |
| et al., | (Hybrid) | | | | | | | | | | | | |
| 2002 | | | | | | | | | | | | | |
| Present | TN:ICSI | Fresh | 204 (7) | 182 | 171 | — | — | 171 | (14) | 107 (52.5) | 23 | 18 | 11.3 |
| invention | (Hybrid) | | | | | | | | | | | | |
| | TN:ICSI | Fresh | 94 (2) | 84 | 77 | — | — | 77 | (6) | 45 (47.9) | 4 | 4 | 4.3 |
| | (Inbred) | | | | | | | | | | | | |
| | TN:ROSI | Fresh | 120 (5) | 108 | 86 | — | — | 86 | (7) | 31 (25.8) | 5 | 5 | 4.2 |
| | (Hybrid) | | | | | | | | | | | | |

^{a}Calculated from number of surviving oocytes
^{b}11 surviving 1-cell embryos were also transferred
^{c}17 surviving 1-cell embryos were also transferred
— Data not available
2 + C# Embryos at 2 to 8 cell stage
m Morula
b Blastocyst

The data in Table 3 is a summary of various micromanipulations. Panel A represents the combined data from seven ICSI microinjection repetitions with approximately an average of 29 oocytes per repetition and attempts with two inbred mouse strains with an average of 47 oocytes per repetition. Such TN:ICSI attempts result in the production of live transgenic pups. Panel B of Table 3 exhibits ROSI microinjection-generated data, with an average of 24 oocytes per microinjection attempt. Each attempt results in a live born transgenic pup, giving a total of five such animals (FIG. 15, B). Panel C of Table 3 depicts pronuclear microinjection attempts and Panel D contains cytoplasmic microinjection data.

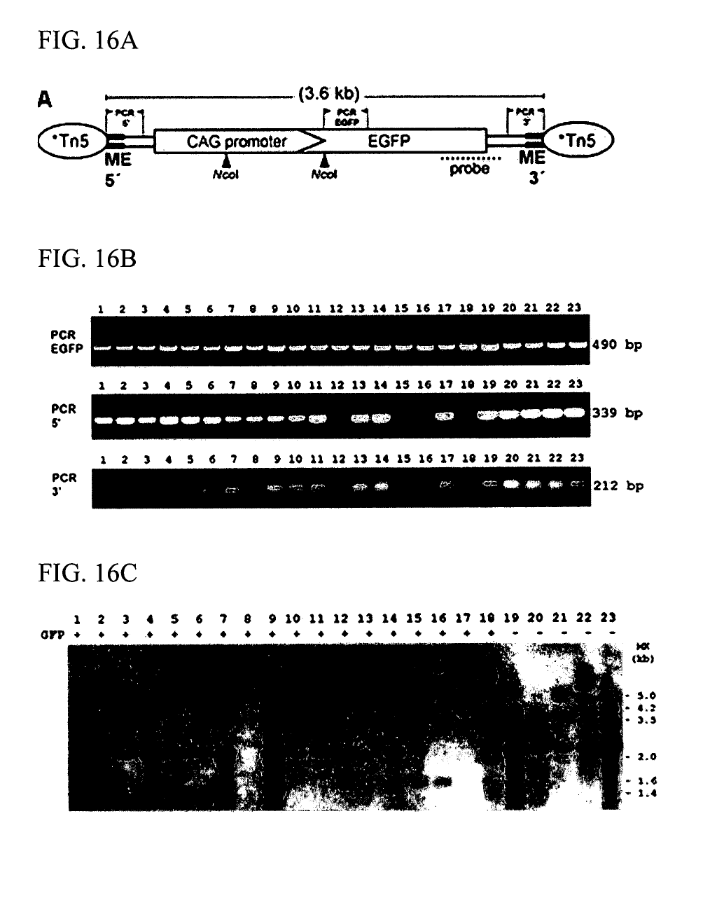

Live born pups are screened by PCR for EGFP transgene integration with primers indicated in FIG. 16A. The ones found to be positive for the transgene are further challenged for full length transgene insertion (FIG. 16B) and their genomic DNA is subjected to Southern blotting to identify the transgene copy number (FIG. 16C). In this case, 23 PCR-positive for EGFP, F0 B6D2F1 hybrid animals can be confirmed for transgene integration (FIGS. 16, B and C). Fragments corresponding to perfectly preserved 5′ and 3′ ends of the transposome are detected in 22 of 23 animals, indicating a high degree of transgene preservation prior to integration, probably due to protection of DNA ends by bound transposase molecules (FIG. 16B). In this case, the transgene number per animal ranges from 1 to ˜20 with 6 out of 23 animals carrying just 1 or 2 copies of the transgene (FIG. 16C, lanes 1, 2, 3, 8, 13 and 17). Lanes 4, 7, 9, 14, 19, 20, 22 and 23 of FIG. 16C additionally contain a strong band in the region of 2.4 kb that resembles concatemerized fragments produced from head to tail integration. Three lanes (12, 15, and 16, FIG. 16C) depict insertions demonstrating a similar pattern and suggest the possible existence of common insertion sites for *Tn5p in the mouse genome. Insertion site analyses with rescue plasmids can elucidate this question and lead to a better understanding of the transposition reactions for *Tn5p in mammals. Such insertion site preferences for target DNA have been demonstrated in bacterial transposition experiments (Goryshin I Y, et al., Proc Natl Acad Sci USA 95: 10716-10721 (1998)).

Transgenesis success with TN:ICSI indicates that *Tn5p-mediated transgenesis by ROSI can also be successful. Round spermatids, the smallest cells in the testis, are easily recognized by their small size and centrally located chromatin mass. Tn5 Transposomes are co-injected with a round spermatid into the cytoplasm of an artificially activated mature unfertilized oocyte. In one trial, this approach (TN:ROSI) results in 5 transgenic EGFP-expressing pups (Table 3, Panel B) corresponding to transgenesis efficiencies of 4.2% of and 16.1% ab. Southern analysis done on the first three born F0 TN:ROSI animals reveals a presence of 1, 7 and 10 copies of the transgene, respectively (FIG. 15A). Southern analysis of genomic DNA blots obtained from biopsies of TN:ROSI F1 progeny mirrors the transgene insertion patterns of the parents.

Tn5 transposomes can also be injected into the pronuclei or the cytoplasm of single-cell embryos of B6D2F1 hybrid mice (Table 3, Panels C and D). Somewhat surprisingly, neither pronuclear nor cytoplasmic injection of transposomes into single celled embryos results in efficient transgenesis (Table 3, Panels C and D).

### Example 10

### Generation of Non-Mosaic Transgenic Mice Via Transposon Microinjection of Oocytes Pre-Expressing PiggyBac Transposase

To overcome the mosaicity of transgenic offspring produced using the method described in Example 1, the piggyBac transposase gene can be inserted into the mouse genome under the control of an oocyte developmental promoter, such as the zona pellucida glycoprotein 3 promoter (ZP3). This generates mice in which the gene for piggyBac transposase is expressed in developing oocytes in females, and upon microinjection of such oocytes with a donor plasmid containing a piggyBac transposon, the transposase is pre-expressed and available to immediately excise and insert the transgene into genomic DNA before the first cell division. To generate such mice transgenic for the piggyBac transposase gene under an oocyte developmental promoter, plasmid pMMK-1 or pMMK-2 are engineered to carry within their piggyBac transposon region the gene for piggyBac transposase under control of the ZP3 promoter, in addition to the gene for EGFP. Ten microliters of this plasmid, at 200 nanograms/microliter, is mixed with 10 microliters of fresh swim-up sperm solution. Each sperm head that has its tail removed in the mixed solution is individually microinjected into a metaphase II (MII) arrested matured mouse oocyte (intra-cytoplasmic sperm injection, ICSI).

Alternatively, a piggyBac donor plasmid engineered to carry the gene for piggyBac transposase within its transposon is incubated with helper plasmid encoding piggyBac under the control of a CAG promoter, mixed with sperm solution and microinjected into a metaphase II (MII) arrested matured mouse oocyte as described above. Two-cell embryos are transferred into the oviducts of pseudopregnant females which are mated with vasectomized males the night before. The females are allowed to give birth to their own young and the newborn pups are examined for EGFP expression in their skin by epifluorescence (FIG. 9). Transgenic offspring are verified by genotyping (as described in Example 9), and grown to reproductive age. Metaphase II arrested matured oocytes from transgenic females are isolated and examined visually for EGFP fluorescence, indicating the presence of the chromosomally integrated transposon in the female's germline cells. Such females are mated with transgenic males to produce offspring that are homozygous for the piggyBac/EGFP transgene.

To then perform a new cycle of transgenesis using piggyBac-expressing oocytes, oocytes are isolated from female mice homozygous for the piggyBac transposase-encoding transgene, and microinjected using ICSI with transposon donor plasmid containing a new gene to be introduced. This gene can be an alternate fluorescent reporter protein such as DsRED to verify transgenesis using DsRED epifluorescence.

### Example 11

### Chimeric PiggyBac and Mos1 Transposases Containing a GAL4 DNA Binding Domain were Able to Perform Transgene Integration in a Site-Directed Manner

Transposon insertion into a functional gene can inactivate the gene, and insertion near regulatory sequences can alter transgene or endogenous gene activity. Methods of integrating transposons at predefined sites were designed to facilitate the appropriate expression of the transgene, and thus, avoid side effects. Briefly, the Gal4 DNA binding domain (DBD) was fused to the Mos1 and piggyBac transposases. Fusion of the Gal4 DBD and each transposase was designed to bring the transposase and associated transgene to a specific upstream activating site (UAS) that was engineered into a target plasmid and was recognized by the Gal4 DBD, thereby targeting transgene insertion to this site. Results of plasmid-based transposition assays in Aedes aegypti embryos demonstrated the efficiency of Gal4-Mos1 and Gal4-piggyBac chimeric transposases.

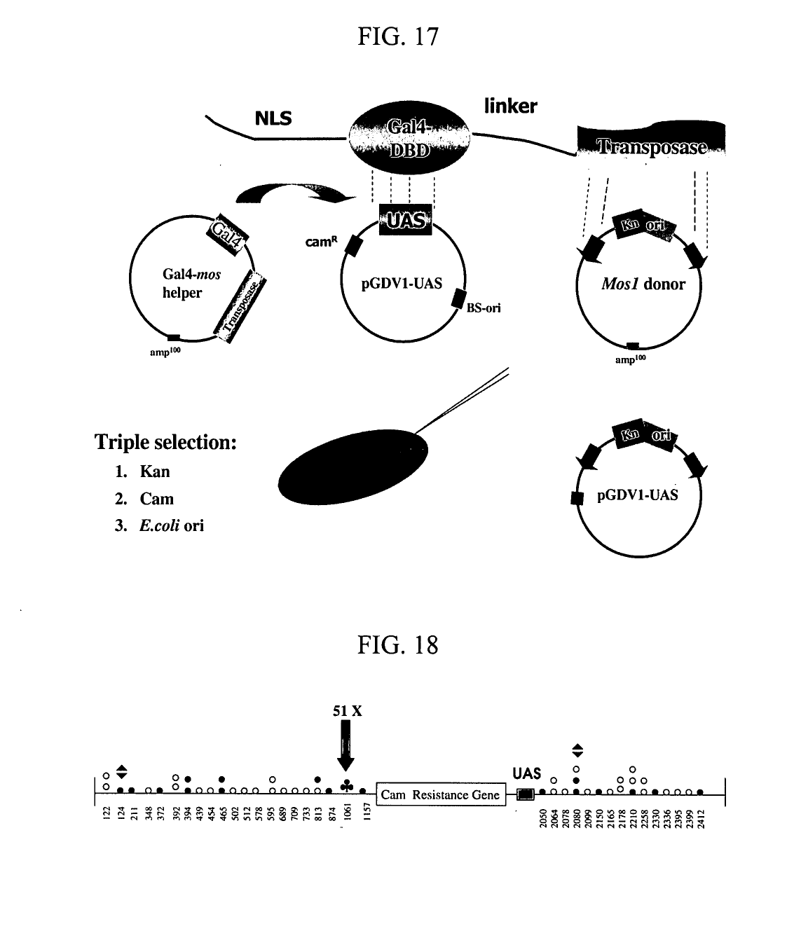

A standard transposition assay was performed with two different helper plasmids, pIE1-Gal4-Mos1 (0.25 μg/ml) or pIE1-Gal4-pB (0.25 μg/ml) (FIG. 17, “Gal4-mos hleper” at left) in Aedes aegypti (Liverpool strain) embryos in individual experiments. Embryo injections were given with a mixture of pGDV1-UAS target (0.5 μg/ml), the pBSMOSoriKan or pB[KO alpha] donor plasmid (0.25 μg/ml), along with the respective helper plasmid into preblastoderm embryos within 2 hours of oviposition. The transposition assay was performed as described previously (Coates, C. J. et al., Gene [1999] 226:317-325). Control transposition assays were also performed using the chimeric transposases and an unmodified pGDVI target plasmid that lacked the UAS target.

Candidate transposition product clones were analyzed by DNA sequencing with the ABI Prism Bigdye terminator cycle sequencing ready reaction kit, following the manufacturer's protocols (Applied Biosystems, Foster City, Calif., USA) and previously described primers (Thibault, S. T., et al., Insect Mol. Biol. [1999] 8:119-123; and Coates, C. J., et al., Mol. Gen. Genet. [1997] 253:728-733.) Reaction products were resolved on an Applied BioSystems automated DNA sequencer (model #ABI3100) and sequence reads analyzed using the Vector NTI suite software (InforMax, North Bethesda, Md., USA).

Plasmid-based transposition assays were performed in Ae. aegypti embryos (FIG. 17). Potential transposition product clones were subjected to BamHI digestion to identify transposition events. The pGDV1-UAS plasmid shown in FIG. 17 was 2.727 kb in size and had a unique BamHI restriction site at nucleotide position 2000. The Mos1 donor element was 4.2 kb, also with a single BamHI site. Thus, the combined molecular weight of any restriction fragments from a transposed element was expected to be 6.9 kb. Putative transposition products with the expected digestion pattern were selected for DNA sequence analysis. The results revealed the duplication of a TA insertion site, the hallmark of Mos1 transposition, which indicates that transposition of the Mos1 element successfully occurred. The transposition frequency for three replicate experiments was calculated by dividing the number of transposition events by the total number of recovered donor plasmids. As a control, a pGDV1 target plasmid lacking a UAS target site was used, such that the Gal4-UAS interaction was absent. The transposition assay results revealed a 12.7-fold increase in transpositional activity over the controls where the UAS target site was absent (Table 5). The transposition frequency was almost 20-fold higher when compared with another control where a regular helper plasmid was used. In addition to the enhanced transposition frequency, the Mos1 chimeric transposase showed a high degree of insertion specificity compared with control experiments.

TABLE 5
Transposition assay data from the use of chimeric
transposases and modified target plasmids.

| | | Total | | |
|---|---|---|---|---|
| | Total donor | number of | Transposition | Fold |
| Experiment | plasmids recovered^{a} | transposition events | frequency^{b} | increase^{c} |
| Control Mos | 4.59 × 10⁶ | 68 | 1.48 × 10⁻⁵ | — |
| helper + pGDV1 | | | | |
| Gal4-Mos helper + | 2.08 × 10⁶ | 48 | 2.32 × 10⁻⁵ | 1.6 |
| pGDV1 | | | | |
| Gal4-Mos helper + | 1.80 × 10⁵ | 53 | 2.96 × 10⁻⁴ | 20 (12.7) |
| pGDV1-UAS | | | | |
| Gal4-piggyBac | 6.12 × 10⁵ | 12 | 1.96 × 10⁻⁵ | — |
| helper + pGDV1 | | | | |
| Gal4-piggyBac | 2.93 × 10⁵ | 67 | 2.28 × 10⁻⁴ | 11.6 |
| helper + pGDV1-UAS | | | | |

^{a}The total number of donor plasmids recovered was estimated by the number of Amp-resistant colonies recovered.
^{b}The transposition frequency was calculated by dividing the number of confirmed transposition products recovered by the total number of donor plasmids recovered. These data were the cumulative data from 3 independent injection and recovery experiments.
^{c}Fold increases for the Mos1 experiments were relative to the control Mos helper + pGDV1 experiment except for the number in parentheses, which represents the fold increase in transposition when using pGDV1-UAS target plasmid compared with the standard pGDV1 target plasmid. The fold increase for the piggyBac experiment was based on the increase observed when using the pGDV1-UAS target plasmid compared to the standard pGDV1 target plasmid.

The pGDV1 target plasmid contains 251 potential TA target sites, of which 60 have been previously identified as insertion sites and 191 are unused sites (Coates, C. J., et al., Mol. Gen. Genet. [1997] 253:728-733). The Cam resistance gene contains 77 TA sites, thus insertions into these sites were not likely to be recovered due to the disruption of the resistance gene used for colony selection, leaving 114 unused sites. Control experiments utilizing the chimeric transposase and an unmodified pGDVI target plasmid lacking the UAS target revealed that integration of the donor element occurred randomly at multiple TA target sites (FIG. 18). However, in the presence of the Mos1 chimeric transposase and the modified pGDV1-UAS target plasmid, transposition primarily occurred at the same TA site, position 1061 of the target plasmid, located 954 by from the inserted UAS target sequence (FIG. 18). Remarkably, the chimeric transposase directed integration to this specific site 96% of the time. Among the Gal4-Mos1 mediated transposition events at the 1061 site, 98% were in a 5′-3′ orientation with respect to the Cam resistance gene. In the control experiments, no integrations occurred at the TA site of the target plasmid.

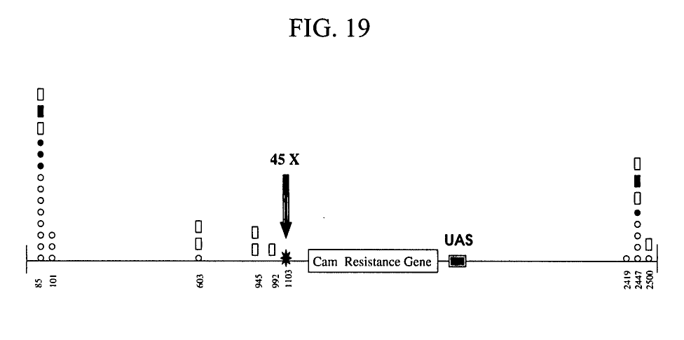

A parallel transposition assay was also performed in Ae. aegypti embryos using the pIE1-Gal4-pB helper. Putative transposition products were selected based on BamHI digestion. The piggyBac donor element was 5.5 kb with a single BamHI site, and thus the transposition product was expected to be 8.22 kb. Actual transposition products were confirmed using DNA sequence analysis. The sequence results revealed the duplication of a TTAA insertion site; the hallmark of piggyBac transposition, thus confirming that piggyBac mediated transposition had occurred. The transposition frequency was 11.6-fold higher compared to the controls. Moreover, in the presence of the piggyBac chimeric transposase and the modified pGDV1-UAS target plasmid, 67% of transpositions occurred at position 1103 site of the target plasmid, located 912 by from the inserted UAS target sequence (FIG. 19). In the control experiments, no integrations occurred at position 1103 of the target plasmid.

The pGDV1 target plasmid contained 29 potential TTAA target sites, of which 8 were in the Cam resistance gene, from which insertions cannot be recovered in this assay (Thibault, S. T., et al., Insect Mol. Biol. [1999] 8:119-123; Lobo, N., et al., Mol. Gen. Genet. [1999] 261:803-810; Li, X., et al., Insect Mol. Biol. [2001] 10: 447-455). Control experiments utilizing the chimeric transposase and an unmodified pGDVI target plasmid lacking the UAS target revealed that integration of the donor element occurred at multiple TTAA target sites (FIG. 19). These sites have been previously used by this element for insertion (Thibault, S. T., et al., Insect Mol. Biol. [1999] 8:119-123). Among the Gal4-piggyBac mediated transposition events at the 1103 site, 80% were in a 3′-5′ orientation with respect to the Cam resistance gene.

1. A method of generating a transgenic embryo containing a piggyBac-like transposon, comprising:
a) contacting a nucleic acid containing a transgene flanked by two terminal repeats with one of the group consisting of: a piggyBac-like transposase polypeptide and a nucleotide sequence encoding a piggyBac-like transposase to form a mixture;
b) contacting said mixture with a sperm to form a composition; and
c) introducing said composition into an unfertilized oocyte to form a transgenic embryo, wherein said piggyBac-like transposase catalyzes the integration of the transgene into the genome of said embryo

2. The method of claim 1, wherein said piggyBac-like transposase is encoded by a nucleotide sequence on the same nucleic acid containing the transgene.

3. The method of claim 1, wherein the nucleic acid encoding said piggyBac-like transposase is an mRNA.

4. The method of claim 1, wherein the transgene is under the control of a promoter.

5. The method of claim 1, wherein the transgene is under the control of the CAG promoter.

6. The method of claim 1, wherein said piggyBac-like transposase is a chimeric transposase comprising a host-specific DNA binding domain.

7. The method of claim 6, wherein the host-specific DNA binding domain of said chimeric transposase comprises Gal4 ZFP.

8. The method of claim 7, wherein the host-specific DNA binding domain of said chimeric transposase is optimized for host specificity.

9. The method of claim 1, wherein the transgene comprises a selectable marker or reporter gene.

10. The method of claim 9, wherein the selectable marker or reporter gene is selected from the group consisting of EGFP, luciferase, β-galactosidase, kanamycin resistance gene (neomycin phosphotransferase), hygromycin resistance gene (hygromycin phosphotransferase), and R6K gamma ori.

11. The method of claim 6, wherein the host-specific DNA binding domain of the chimeric transposase is fused to the N-terminus of said transposase.

12. The method of claim 6, wherein the host-specific DNA binding domain of the chimeric transposase is fused to the C-terminus of said transposase.

13. A method of generating a transgenic animal, comprising implanting, into a viable mother, an embryo generated according to claim 1.

14. The method of claim 13, wherein said mother is a vertebrate.

15. A method of generating a transgenic animal, containing in the genome of one or more of its cells a piggyBac-like transposon, the method comprising:
a) contacting a nucleic acid containing a transgene flanked by two terminal repeats, with a sperm to form a mixture;
b) introducing said mixture into an unfertilized oocyte from a transgenic female containing in its genome a piggyBac-like transposon encoding a piggyBac-like transposase under the control of an oocyte developmental promoter, thus forming a transgenic embryo, whereby said transposase is expressed in the oocyte and catalyzes the integration of the transgene into the genome of said embryo; and
c) implanting said transgenic embryo into a viable mother.
